(19) United States
(12) Patent Application Publication
(10) Pub. No.: US 2010/0286761 A1
(43) Pub. Date: Nov. 11, 2010

(54) Translucent Outer Sheath

(76) Inventor: Jason Benjamin Wynberg, Oak Park, MI (US)

Correspondence Address: Jason Wynberg, 1853 61st Street, Brooklyn, NY, 11204, US

(21) Appl. No.: 12/753,019

(22) Filed: Jul. 12, 2010

Related U.S. Application Data
(60) Provisional application No. 61/165,834, filed on Apr. 1, 2009.

(51) Int. Cl. A61F 2/84

(52) U.S. Cl. 623/1.23

(57) ABSTRACT

A transparent outer covering used in a co-axial metallic ureteric stent for deployment of the lower tip of the metallic ureteric stent in relation to the ureteric orifice. Exact positioning of the stent is critical to avoid complications and ensure proper functioning of the stent. Stent is deployed from within outer sheath in a coaxial system. The function of the coaxial outer sheath is to provide a straight channel for deployment and positioning of the curl-tipped metal stent (double-J stent). Without the outer sheath it would simply not be possible to advance the flexible, curl-tipped metal stent into the kidney. Surgeon controls a ureteric catheter ‘pusher’ whose tip is advanced against the lower tip of the metal stent within the OPAQUE sheath, Deployment is achieved by withdrawing the OPAQUE outer sheath, while holding stent/ureteric catheter in static position. The lower tip of the metal stent must be deployed between the bladder neck and the ureteric orifice which are soft tissue structures and therefore not visible on flouroscopy. The surgeon absolutely requires direct visualization of the lower tip of the stent to see its relation to these landmarks. The existing outer deployment sheath is opaque. Converting outer deployment sheath to translucent would thereby substantively improve deployment accuracy of stent and avoid complications of deployment above ureteric orifice or below bladder neck.

This application emanates from a previous filing dated Apr. 1, 2009; application No. 61165834

### FIELD OF THE INVENTION

This invention relates generally to stents, and particularly to the ureteric stent in relation to the ureteric orifice.

### BACKGROUND OF THE INVENTION

Placement of a metallic double-pigtail ureteric stent is performed in cases of refractory ureteric stricture disease, either benign or malignant. A commonly used device employs a co-axial system to place the stent. First, a guidewire is placed across the narrowing. A co-axial duo of an inner 5 French diameter ureteric catheter and outer 9 French diameter OPAQUE PLASTIC sheath is passed over the wire under cystoscopic and fluoroscopic guidance above the level of obstruction. Both the guidewire and inner 5 French ureteric catheter are then removed, leaving only the 9 French diameter sheath above the obstruction. The metal stent is then loaded into the sheath, and the 5 French ureteric catheter is used to ‘push’ the metal stent thru the 9F OPAQUE PLASTIC sheath into the kidney, above the obstruction. Final deployment of the metal stent involves holding the inner 5 French ureteric catheter in a fixed position (in order to hold the metal stent in a fixed position), and slowly withdrawing the outer OPAQUE sheath. First, the sheath uncovers the upper pigtail in the kidney. As the sheath is further withdrawn, attention of the surgeon turns to the exact location of the lower tip of the metal stent, specifically its relationship to the ureteric orifice and bladder neck, as final deployment of the distal tip of metal stent must be between these two landmarks.

The invention herein is to be able to see through the end of the 9 French plastic sheath i.e. that it not be opaque, but rather ‘see-through’. By doing so, the surgeon will see the relationship between the lower tip of the stent and the ureteric orifice and bladder neck, reducing the chance of deploying the tip of the stent above the ureteric orifice or below the bladder neck (the critical relationship between ureteric orifice and tip of stent can only be appreciated visually; fluoroscopy does not demonstrate the ureteric orifice or bladder neck. Again, this invention submission is to use a transparent/translucent 9 French outer sheath in a coaxial stent placement system to aid in deployment of the distal tip of a double pigtail ureteric stent, to avoid complete intra-ureteric deployment of the stent or urethral deployment i.e. below bladder neck.

Flouroscopy and cystoscopy-assisted placement of a metallic, double-pigtail ureteric stent is performed to maintain antegrade ureteric drainage in cases of refractory ureteric obstruction, commonly from dense stricture disease or malignancy. The present co-axial deployment system employs an opaque plastic 9 French outer sheath. This invention replaces this with an outer sheath whose distal 10 cm, or entire length, is transparent or translucent. This change will significantly improve the safety of deployment of the metallic stent, by allowing the surgeon to appreciate in real time the relationship between the distal tip of the metal stent with the ureteric orifice. This information allows the surgeon to avoid deploying the distal aspect of the stent above the level of the orifice, in the distal ureter. The present opaque 9 French sheath blinds the surgeon to the position of the distal tip of the stent, thus increasing the risk of complete intra-ureteric stent deployment. This principle is applied to ALL co-axial deployment systems for ureteric stents, e.g. antegrade systems. Where direct visualization of the ‘physician-side’ end of the stent improves deployment, a transparent/translucent outer sheath replaces an opaque sheath. This considers that flouroscopy cannot provide all soft-tissue anatomy which can provide important landmarks for deployment.

### SUMMARY OF THE INVENTION

It is an object of the invention to provide A transparent outer deployment sheath which allows deployment of the lower tip of the ureteric stent in relation to the ureteric orifice.

The current external coaxial deployment sheath in the Cook (R) Resonance Metallic Stent is opaque. As such, the only way to know where the metal stent is in relation to the ureteric orifice is by xray flouroscopy. This invention replaces the blue opaque outer deployment sheath with a clear deployment sheath.

A transparent outer deployment sheath allows deployment of the lower tip of the ureteric stent in relation to the ureteric orifice. Currently, positioning in relation to the ureteric orifice is NOT knowable, as the ureteric orifice does not show up on Xray. The current blue opaque sheath blocks the view of the position of the lower tip of the metallic stent, so that the surgeon deployes the stent based only on fluoroscopic position. This can lead to erroneous deployment of the stent ABOVE the ureteric orifice (which is not radioopaque). Such would lead to more invasive surgery to remove or reposition the stent. A clear outer sheath would add the visual dimension to the stent deployment

This invention is a substantial improvement over the existing outer sheath. The current version of this stent presumes that deployment is adequate by fluoroscopy alone. This is not the case, and using a clear outer sheath adds the benefit of visual information to know where the stent is being deployed in relation to critical anatomy.

### BRIEF DESCRIPTION OF THE DRAWINGS

FIG. 1: Front view of invention, depicting final stage of deployment, namely the active withdrawl of TRANSPARENT PLASTIC outer sheath from body.

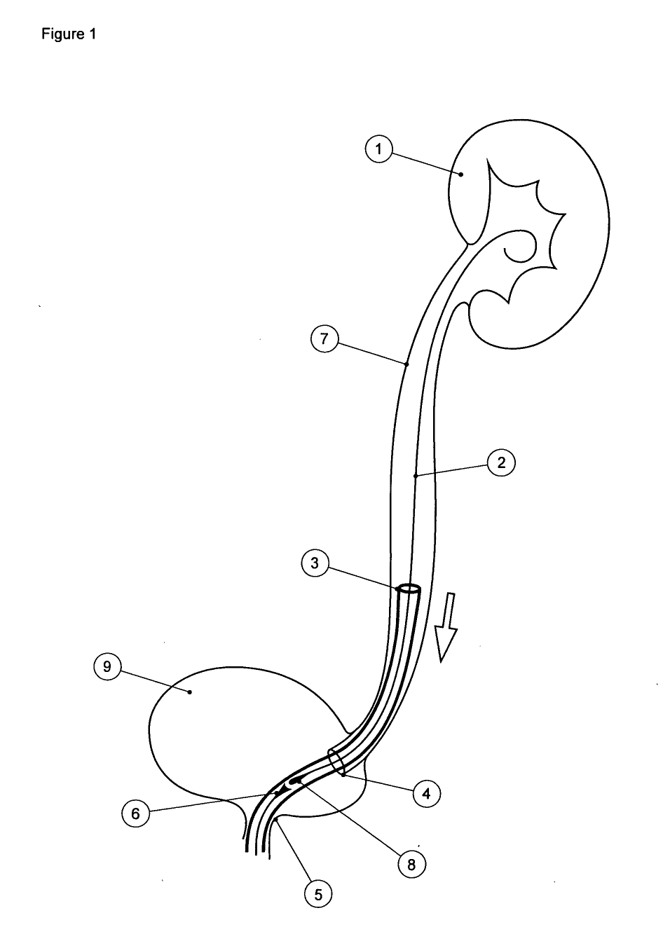

### DETAILED DESCRIPTION OF THE DRAWINGS

1. Kidney—produces urine.

2. Metallic stent—The upper portion of stent demonstrates proper curling in proper deployment position. The lower portion of stent is not yet deployed, as OPAQUE PLASTIC outer sheath is still overlying lower portion of stent, preventing it from curling as seen on the upper portion of stent

3. 9 French TRANSPARENT outer sheath—The subject of this utility submission. This sheath is demonstrated in transparent form, allowing surgeon to view distal tip of metallic stent (#8) and its relations to critical landmarks, namely ureteric orifice (#4) and bladder neck (#5). If this outer sheath (#3) were opaque, it would not be possible to see (#8). This would result in deploying stent without knowing location of (#8) in relation to #4 and #5.

4. Ureteric orifice—upper limit of deployment of distal tip (#8) of metallic stent. If outer sheath (#3) is removed with tip of metallic stent (#8) ABOVE ureteric orifice, this is a misplacement, causing difficult removal and complications.

5. Bladder neck—lower limit of deployment of distal tip (#8) of metallic stent. If outer sheath (#3) is removed with tip of metallic stent (#8) BELOW bladder neck, this is a misplacement, causing difficult placement and complications.

6. 5 French ureteric catheter, “pusher”. This tip of this catheter abutts the distal tip of the metallic stent (#8). The surgeon holds the end of the 5 French ureteric catheter outside the patient (not demonstrated in illustration). By advancing or withdrawing this 5 French ureteric catheter, the surgeon can adjust the level of the distal tip of the metallic stent (#8). The tip of the metallic stent must lie between the ureteric orifice (#4) and bladder neck (#5) at the moment that the outer sheath (#3) is removed. The action of advancing or withdrawing the 5 French ureteric catheter outside the body will determine the position of the stent tip (#8) and thereby determine the ultimate success of the stent deployment. The surgeon must see this distal tip (#8) under direct vision, which requires a TRANSPARENT outer sheath (#3).

7. Ureter—Tube that drains urine from kidney into bladder, where urine is stored between voids.

8. Metallic stent, distal tip. Once outer sheath (#3) is removed, this distal tip will curl into its fully deployed position, as demonstrated above inside the kidney.

9. Bladder cavity—stores urine between urinary voids.

I claim:

1. A translucent outer sheath device for inserting a stent in a body cavity, said device comprising: an outer elongated mantle having first and second ends; a transparent body throughout having, said first end portion of said member being capable of removably receiving said stent; and means for locating the position of said stent within said body cavity.

2. A translucent outer sheath device for inserting a stent in a body cavity, said device comprising: a translucent body wherein direct visualization to flouroscopy is achieved to determine location of lower tip of stent in relation to critical deployment landmarks.

3. A translucent outer sheath of claim 1 wherein said locating means comprises a translucent body wherein direct visualization to flouroscopy is achieved to determine location of lower tip of stent in relation to critical deployment landmarks.

4. A translucent outer sheath of claim 2 wherein said locating means comprises a translucent body wherein direct visualization to flouroscopy is achieved to determine location of lower tip of stent in relation to critical deployment landmarks.

5. A translucent outer sheath device wherein the outer sheath of
a coaxial endoscopic deployment system being transparent/translucent.

6. A translucent outer sheath of claim 5 wherein said locating means comprises a translucent body wherein direct visualization to flouroscopy is achieved to determine location of lower tip of stent in relation to critical deployment landmarks.
